(19) United States
(12) Patent Application Publication
(10) Pub. No.: US 2010/0015076 A1
(43) Pub. Date: Jan. 21, 2010

(54) POLYMER STRUCTURE REACTING WITH KERATIN, SYNTHESIS AND USE THEREOF

(75) Inventors: Matteo Zanotti Russo, Rimini (IT); Nicola Barbini, Legnano (IT)

Correspondence Address: HESS PATENT LAW FIRM, P.C., 9 MIRAMAR LANE, STAMFORD, CT, 06902, US

(73) Assignee: EUDERMIC S.R.L., Legnano (MI) (IT)

(21) Appl. No.: 12/158,320

(22) PCT Filed: Dec. 23, 2005
(86) PCT No.: PCT/IT05/00764

§ 371 Date: Aug. 6, 2008

(51) Int. Cl. A61K 8/81; C08F 120/56; C08F 26/06; C08F 136/00; C08F 220/42; C08F 118/02; C07C 233/00; C07D 207/30; C07C 255/07; C07D 209/82; C07C 69/52

(52) U.S. Cl. 424/70.11; 526/303.1; 526/263; 526/335; 526/297; 526/319; 526/260; 526/346; 564/204; 548/560; 558/462; 548/440; 560/205; 424/78.02

(57) ABSTRACT

Novel (co)polymeric structures having functional groups making possible, under conditions that are safe for human use, the formation of chemical bonds with human keratin, and in particular with the keratin present in hair, eyelashes and eyebrows, skin, nails, lips, the oral mucosa and tissues of the external genital organs are disclosed; the invention also relates to the synthesis of such (co)polymeric structures and the use thereof in the fields of cosmetics and medicine.

### FIELD OF THE INVENTION

The present invention relates to polymeric structures reacting with keratin, their synthesis and the application and use thereof.

More particularly, the present invention concerns novel (co)polymeric structures having functional groups making possible, under conditions that are safe for human use, die formation of chemical bonds with human keratin, and in particular with the keratin present in hair, eyelashes and eyebrows, skin, nails, lips, the oral mucosa and tissues of the external genital organs; the invention also relates to the synthesis of such (co)polymeric structures and the use thereof in the fields of cosmetics and dermatology.

The (co)polymers according to the invention may in themselves constitute and hence perform a structural, protective, aesthetic, reconstitutive role or may constitute the substrate for attaching other functional agents to the keratin surface, such as protective agents such as antioxidants or antibacterial agents, or functional agents, such as for example chemical structures having volumising, lubricant, detangling, brightening, dyeing, antibacterial, protective and such like actions.

### DESCRIPTION OF THE PRIOR ART

In recent years, much of the research relating to products for nails, hair and skin envisages, among the primary aims, the necessity to implement the interactions of materials, substrates, active ingredients or dyes with the keratin matrices which, with certain differences, are common to the external body surfaces and hair. Indeed, many of the cosmetic functions are associated with both the modification of surface aspects and the need to carry or fix certain functional structures into the keratin surface, through both mechanical and chemical functions. Among the functions undergoing development, the following may be mentioned:
the film forming power of hair conditioners
hairstyling fixatives
film forming protective agents for hair, skin and nails
water and abrasion resistance of sun products
the fixing power of colourants and pigments to nails (enamels), hair (dyes), skin (make-up)

While products intended for nails primarily have the aim of colouring or applying supporting structures for reinforcing the nails, products intended for skin and hair have a much wider range of functions and functional goals, which, in any case, very frequently involve the need to modify the surface structures or to apply molecules with appropriate functionality to them. In the trichological field, for example, the development and use of filmogens, with the declared aim of increasing the permanence of the structures applied onto the keratin surface by as much as possible, has been very significant, just as with skin products, sun products that are resistant to water or “barrier effect” creams have been developed.

EP1192932 describes the development of a hair fixing gel for hairstyling with greater fixative resistance and increased resistance to humidity. This performance is obtained thanks to the film forming characteristics of the polymer and the interactions between the polymer structure and the keratin surface. Indeed, the film forming power increases the contact surface between the polymer and the keratin, and emphasises the efficacy of the Van der Waals interactions and the dipole-dipole interactions or the hydrogen bonds guaranteeing the adhesion of the polymer.

US2004146471 relates to a composition having high fixing power and high resistance due to the use of 3 film forming polymers.

The need to fix and functionalise polymer substrates is comprehensively disclosed in EP1525877, which claims the capacity to fix hairstyles and hold them for long periods of time thanks to the use of polymers which modify their structure as a function of temperature.

US2003135004 concerns a polymer composition which, thanks to the peculiar structures, guarantee adhesion and resistance by interacting with keratin; their use for fixing dyes onto the skin of the face (for use in makeup products), nails, hair, eyelashes or eyebrows is disclosed.

Among the functional ingredients to be fixed to keratin substrates, pigments and dye substances in general may be mentioned. The colouring of cosmetic products, just as the capacity of cosmetic products to colour skin, nails and hair are regulated in both Europe and in the USA, and in the majority of countries, so that the useable colourants are disclosed in positive lists, such as those disclosed in the enclosures to Italian Law 713/86 and wash. Therefore, there is a need to create formulations for use on hair, capable of having a useful lifespan, longer than possible at present.

### SUMMARY OF THE INVENTION

The aim of the present invention is to solve the above discussed problem, and to provide formulations for the treatment of hair in particular, and body hair, nails and skin in general, capable of lasting over time.

This aim is achieved by means of the present invention which concerns a (co)polymer for cosmetic use, characterised according to claim 1.

More particularly, it is an object of the invention a class of (co)polymers which may find application in the cosmetic and dermatological sectors, and particularly in the formulation of products for the care and treatment of hair, eyelashes, eyebrows, body hair, nails, skin, lips, mucosa and external genital organs, which are characterised by the presence of reactive functional groups capable of reacting with keratin under mild reaction conditions and in the presence of solvents and reagents suitable for use on humans.

The polymers and copolymers of the invention have general formula

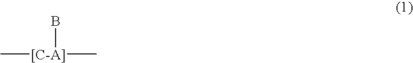

wherein [C-A] is the polymeric structure and B is a functional group capable of reacting with keratin, B being selected from —SH and leaving groups capable of being substituted by the amine groups from lysine European Directive 76/768/EEC. Hence, because it is not possible to easily modify the pigmenting agents, research aimed to develop substrates, mostly film forming, through increasing their anchoring and resistance to abrasion, as e.g. described in EP0960617 Composition for dying keratinous fibres with a direct cationic dye and a sustaining polymer.

EP1321126 (Cosmetic compositions comprising polymers comprising complementary chemical functional groups) describes and claims the use of two complimentary polymers which react to form covalent bonds between one another, only when they are applied on the keratin, thus forming a film that is extremely resistant to rinse and abrasion. In this case, anchoring to the keratin is implemented thanks to the formation of efficient macromolecular structures in situ which allow increased interactions with the keratin, and consequent non-covalent adhesion.

In WO2004098488 (Use of dithiols in a hair-perming composition) water soluble dithiols comprising a C₃-C₁₀ chain between the two thiol groups are used as reducing agents in place of the thioglycolic acid and derivatives thereof, in order to obtain the main advantage of obtaining the “permanent” hair-wave effect (the rupture of the disulphide bridges present in the keratin and their re-establishment “in a wave state”) under favourable pH conditions and using relatively odour-free reagents.

The problem with the above disclosed formulations and systems is that the structures formed have short lifespans, and in some cases do not even survive one residues present in the keratin structure.

According to one aspect of the invention, the macromolecular polymer to be anchored to the keratin by means of covalent bonds has additional groups, useful for attaching functional groups such as: dyes, antioxidants, brighteners, volumisers, sun screens, antibacterial agents, protective agents, pigments.

An additional object of the indention are cosmetic formulations containing such a polymeric matrix, and the conditions and methods under which said innovative cosmetic product are used. Dermatological formulations containing the polymers and copolymers of the invention, and the use of said polymers and copolymers in the field of medicine, and dermatology in particular, are equally objects of the invention.

Another object of the invention is the synthesis of (co)polymers suitable to the uses outlined above.

According to a first aspect of the invention, the reactive group of the polymer, i.e. the group which must react with a functional group of the human keratin, is a leaving group capable of being substituted on the polymer by an amine group from a lysine residue in the keratin. With such functional groups, the polymer-hair bond is generally irreversible.

According to another aspect of the invention, the reactive group of the polymer according to the invention is a thiol group (—SH) which reacts with the thiol groups deriving from the cystein residues, of which hair is rich. With this functional group, the polymer-hair bond is reversible.

The two functional groups discussed above will form covalent bonds, even though through different mechanisms. The main advantages deriving from the use of thiol groups lie in the ease with which they may be chemically reduced and hence cleaved, thus giving a polymer capable of being bound to the keratin structure, and subsequently removed from the same.

Indeed, the thiol groups may be bound to homologous thiol groups present on hair, according to the known mechanism with which through internal homolysis-oxidation a “perm” may be achieved through the alternating use of reducing agent (thioglycolic acid) and oxidising agents (hydrogen peroxide).

Indeed, the thiol groups may be bound to homologous thiol groups present on hair, according to the well known mechanism with which through internal homolysis-oxidation a perm may be achieved through the alternating use of reducing agent (thioglycolic acid) and oxidising agents (hydrogen peroxide) according to the-scheme:

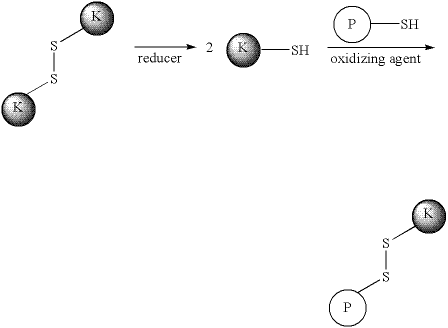

Polymers which may be used for binding to keratin by means of disulphide bridges, forming the subject of the present invention, are characterised by the presence of ah SH group in the side chain and may be synthesised according to the following scheme:

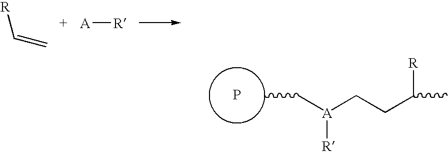

wherein R′=Cl, Br, I, OH

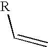

=acrylic and methacrylic, itaconic acid and the methyl methyl and ethyl esters thereof, with a C₃-C₃₀ alkyl group, acryloyl morpholine and derivatives thereof, styrene, styrene with halogen groups in the o,m,p positions and derivatives thereof, vinyl chloride and derivatives thereof, vinyl acetate and derivatives thereof, acrylamide and derivatives thereof, N,N dimethylacrylamide and derivatives thereof, N,N diethylacrylamide and derivatives thereof, acrylamide substituted with C₃-C₃₀ alkyl groups, vinylpyrrolidone and derivatives thereof, butadiene and derivatives thereof, cyanoacrylates and cyanomethacrylates substituted with C₃-C₃₀ alkyl groups, furoic acid and derivatives thereof, vinyl carbazole and derivatives thereof, vilidene chloride and derivatives thereof, vinyl alcohol and derivatives thereof, terpenes and modified terpenes.

An acrylic and methacrylic, itaconic acid and the methyl and ethyl esters thereof, with a C₃-C₃₀ alkyl group, acryloyl morpholine and derivatives thereof, styrene, styrene with halogen groups in the o,m,p positions and derivatives thereof, vinyl chloride and derivatives thereof, vinyl acetate and derivatives thereof, acrylamide and derivatives thereof, N,N dimethylacrylamide and derivatives thereof, N,N diethylacrylamide and derivatives thereof, acrylamide substituted with C₃-C₃₀ alkyl groups, vinylpyrrolidone and derivatives thereof, butadiene and derivatives thereof, cyanoacrylates and cyanomethacrylates, furoic acid and derivatives thereof, vinyl carbazole and derivatives thereof, vilidene chloride and derivatives thereof, vinyl alcohol, terpenes and modified terpenes, having a halide or hydroxyl group as a substituent.

Suitable activated linear or cyclic silicone polymers, polysaccharides and naturally derived polymers lending themselves to the same functionalisation and the same aim, may be easily functionalised using thiol or amide active groups disclosed hereinafter for the aim, object of the present invention, of binding the polymer structure covalently to hair.

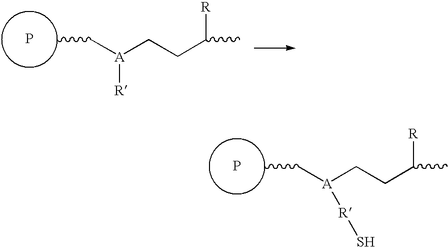

The following table lists some of the compounds that may be used in the synthesis of the polymers according to the invention.

TABLE 1

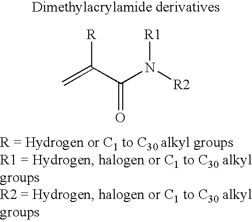

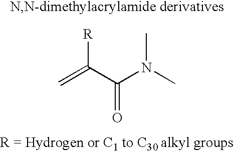

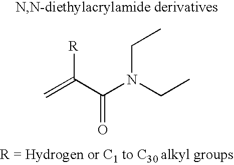

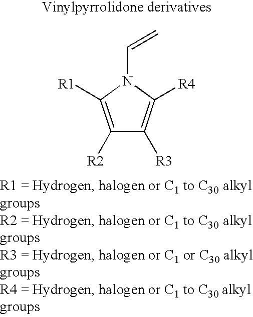

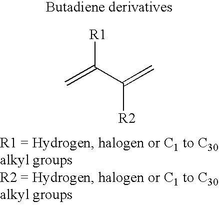

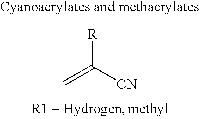

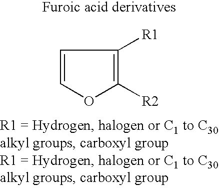

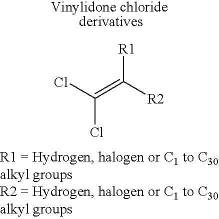

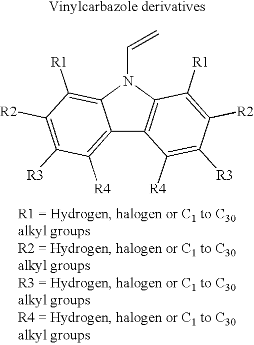

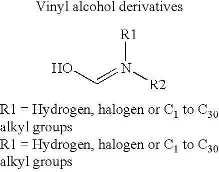

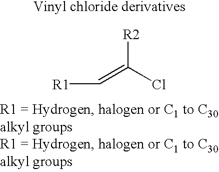

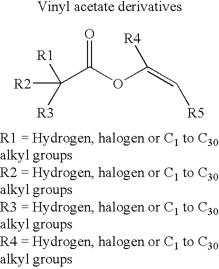

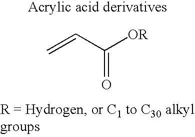

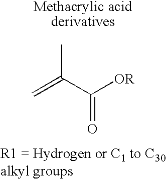

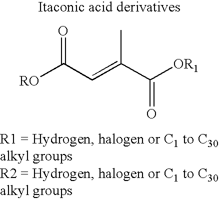

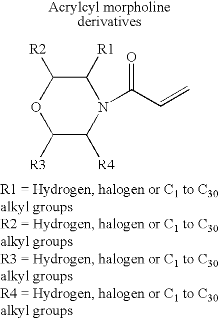

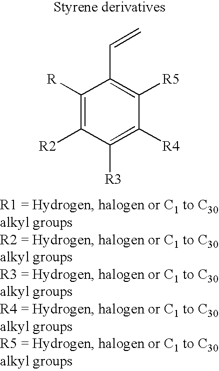

The main drawback in the formation of polymer-keratin thiol bonds lies in the conditions under which such bonds are formed, which are the same as those under which the hair perm or tint is obtained. This approach, despite allowing the attainment of a reversible bond, is however limited to use on hair, and without obtaining any significant advantages from the toxicological viewpoint with respect to the conditions of use necessary for the application of dyes by oxidation, or perms.

The advantage of forming covalent bonds using the amine groups of lysine, is that of being able to operate under milder conditions.

As is known, amine groups may easily contribute to the formation of covalent bonds through nucleophilic substitution on groups containing a partially positively charged C atom. The presence of leaving groups may strongly promote this reaction, allowing nucleophilic substitution even when operating under “hairdresser” or “domestic” conditions, i.e. at room/body temperature and using harmless solvents, so as to allow safe use both when handled by a professional hairdresser/beautician, and when used by the consumer themselves.

The leaving groups which may be used are those with extremely low potential levels of harmfulness or toxicity, also considering the fact that application is followed by rinsing.

Among the suitable leaving groups that have been identified are included the aromatic amines, and in particular 1,2,3-benzotriazole has been identified, the toxicological profile of which is favourable and suited to the envisaged use.

Other leaving groups include chloride, bromide, iodide, toaylates and derivatives thereof, mesylates and derivatives thereof, brosylates and derivatives thereof, nosylates and derivatives thereof, triflates and derivatives thereof, nonaflates and derivatives thereof, tresylates and derivatives thereof, dicyclohexylcarbodiimide adducts and derivatives thereof, carbonyldiimadazole adducts, 2-chloropyridinium ion adducts, 3-chloroisoxazole ion adducts, 2,2′-dipyridyldisulphide adducts, disulphurodimidazole adducts, BOP-Cl adducts and hydroxysuccinimmide adducts. Table 2 lists the leaving groups adapted to the invention.

TABLE 2

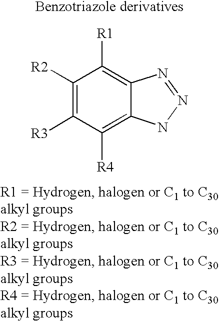

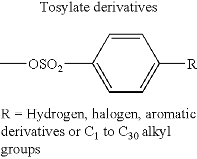

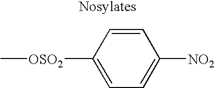

Mesylate derivatives
—OSO₂—R
R = Hydrogen, halogen or C₁ to C₃₀
alkyl groups
Triflate derivatives
—OSO₂—R
R = C₁ to C₃₀ fluoroalkyl
derivatives
Tresylate derivatives
—OSO₂—R
R = Aromatic groups and C₁ to
C₃₀ alkyl groups where one or
more hydrogens are
substituted by one or more
fluorine atoms

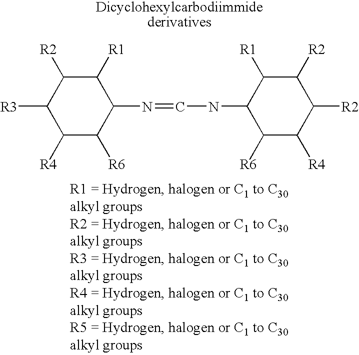

Dicarbodiimmide derivatives
R1—N═C═N—R2
R1 = C₁ to C₃₀ alkyl groups
R2 = C₁ to C₃₀ alkyl groups

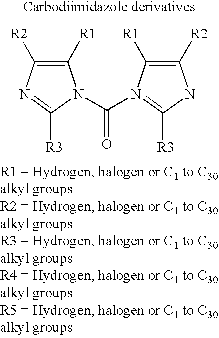

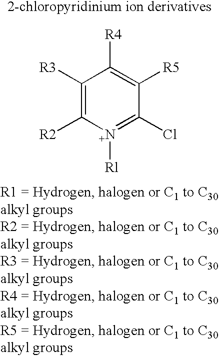

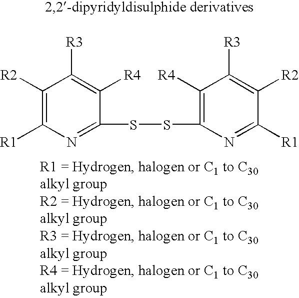

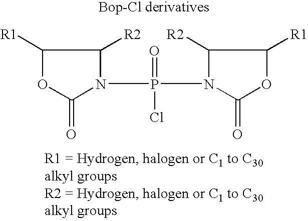

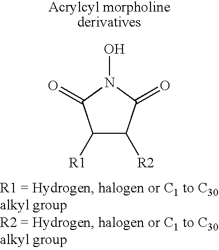

To introduce the leaving groups into the polymers under test, the appropriate monomers have been synthesised having the structure:

A-B (1)

Wherein A is constituted by the following groups: acrylic, methacrylic, itaconic, sorbic, protonic, cyanoacrylates

and

B is given by leaving groups identified as: chloride, bromide, iodide, tosylates and derivatives thereof, mesylates and derivatives thereof, brosylates and derivatives thereof, nosylates and derivatives thereof, triflates and derivatives thereof, nonaflates and derivatives thereof, tresylates and derivatives thereof, dicyclohexylcarbodiimmide adducts and derivatives thereof, carbonyldiimadazole adducts, 2-chloropyridinium ion adducts, 3-chloroisoxazole ion adducts, 2,2′-dipyridyldisulphide adducts, disulphurodimidazole adducts, BOP-C1 adducts and hydroxysuccinimmide adducts, particularly 1,2,3-benzotriazole.

The monomers of structure A-B may be copolymerised (as disclosed below) with other monomer families or polymerised directly onto other polymer structures. Suitably activated linear or cyclic silicone polymers, polysaccharides and naturally derived polymers lending themselves to the same functionalisation and the same aim, may be easily functionalised using thiol or amide active groups (—CO—B) disclosed hereinafter for the purpose of binding the polymer structure covalently to hair.

Suitable leaving groups are reported in table 2.

Such A-B monomers have predominantly been obtained by substitution of the halide derivatives of the acids corresponding to the respective A structures, particularly chlorides. The Cl has been subsequently substituted by the leaving groups B.

The functionalised monomers A-B according to the present invention preferably have the general formula:

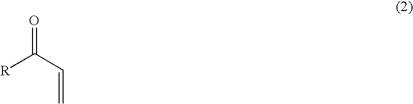

Wherein the R group is: chloride/bromide, iodide, tosylates (and the family thereof), carbonylimidazole derivatives, dicyclohexylurea derivatives (and the family thereof), 2-chloropyridinium ion derivatives, 3-chloroisoxazolidinium ion derivatives, 2,2′-dipyridyldisulphide derivatives, disulphurodimidazole, hydroxysuccinimide, aromatic amines particularly 1,2,3-benzotriazole. In this embodiment R corresponds to the group B.

The A-B monomers thus obtained are polymerised by free-radical polymerisation under nitrogen with known monomers selected depending on the required degree of polymerisation, glass transition temperature of the resulting polymers, the toxicity and the characteristics of the resulting polymer.

In particular, the C monomers are selected from acrylic and methacrylic, itaconic acid and the methyl and ethyl esters thereof, with a C₃ to C₃₀ alkyl group, acryloyl morpholine and derivates thereof, styrene, styrene with halogen groups in the o,m,p positions and derivates thereof, vinyl chloride and derivates thereof, vinyl acetate and derivates thereof, acrylamide and derivatives thereof, N,N dimethylacrylamide and derivates thereof, N,N diethylacrylamide and derivatives thereof, acrylamide substituted with C₃ to C₃₀ alkyl groups, vinylpyrrolidone and derivatives thereof, butadiene and derivatives thereof, cyanoacrylates and cyanomethacrylates substituted with C₃ to C₃₀ alkyl groups, furoic acid and derivatives thereof, vinyl carbazole and derivatives thereof, vilidene chloride and derivatives thereof, vinyl alcohol and derivatives thereof, terpenes and modified terpenes.

Table 1 lists the monomers that may be used for the synthesis of the polymers of the invention.

### BRIEF DESCRIPTION OF THE FIGURES

The invention will now be disclosed in greater detail with reference to the following examples and the figures enclosed by way of non-limiting illustration, wherein.

FIG. 1 is an enlarged view of an untreated hair, and

FIG. 2 is an enlarged view of a hair treated with a polymer according to the present invention.

The synthesis of thiol polymers of the monomers provided with leaving groups and their bonding to keratin through keratin lysine residues, will now be illustrated in further detail with reference to the following examples.

### EXAMPLE 1

### Polymer Synthesis Where B=—SH

Into a carefully flamed Schlenk vial under nitrogen are added 1.156 mmol of methylmethacrylate and 1.156 mmol (176 mg) of p-chloromethylstyrene glycidyl methacrylate dissolved in 3 ml of anhydrous DMF and 4 mg of AIBN. The solution is subsequently transferred, by means of a needle under nitrogen pressure, into a round-bottomed polymerisation flask equipped with a magnetic stirring bar. The reaction is carried out at a temperature of 60° C. with constant stirring. After 20 hours, the polymer obtained, dissolved again in DMF, is precipitated in ethyl ether and filtered.

### Thiol Group Functionalisation

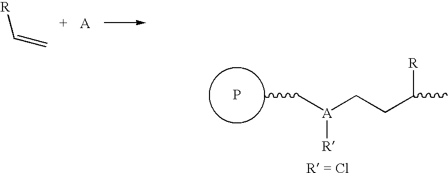

The functionalisation of the side chain with a thiol group (—SH) has been carried out as reported in the literature (Journal American Chemical Society 65 1943 1466, 1467): at 65° C., in ethyl alcohol, with NaSH and SH₂according to the following scheme:

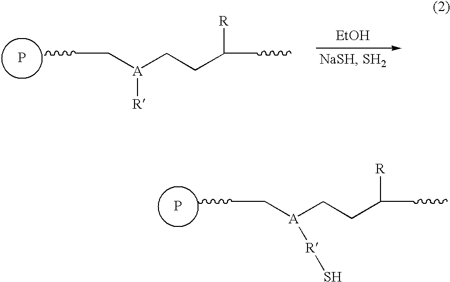

The polymer synthesised according to the method reported above has been used in the following in vitro test (example 4A).

### EXAMPLE 2

### Polymer Synthesis with B=Leaving Group Synthesis of N-Acryloyl-1,2,3 Benzotriazole

Into a carefully flamed 100 ml three-necked round-bottomed flask fitted with a mechanical stirrer, dropping funnel and reflux condenser, under nitrogen are introduced 1.19 ml (14.7 mmol) of acrylic chloride and 10 ml of anhydrous toluene. The reaction mixture is maintained at a temperature of 0° C. and, over a 40 minute period, a mixture consisting of 1.75 g (14.7 mmol) of 1,2,3-benzotriazole and 1.49 g (14.7 mmol) of triethylamine dissolved in 25 ml of anhydrous toluene added. The reaction proceeds as indicated in the scheme reported herein.

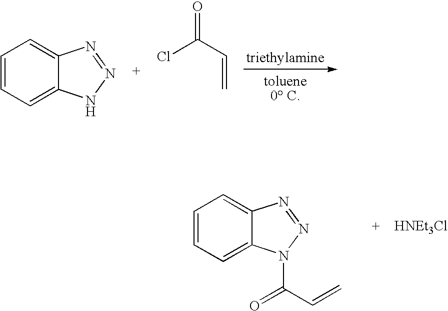

After stirring for a period of 20 hours, the triethylamine hydrochloride formed is filtered, washed with toluene, and the combined toluene phases washed in sequence with water, a saturated sodium chloride solution and water once more, dried over anhydrous sodium sulphate and the solvent eliminated under reduced pressure to give crude N′-acryloyl-1,2,3-benzotriazole which is subsequently purified by silica gel chromatography. Once the solvent is eliminated under, reduced pressure, after a few minutes the product crystallises giving white crystals.

### Polymer Synthesis

The step subsequent to the formation of the monomer is the synthesis of the polymer molecule which will have to maintain its reactivity towards the keratin amino groups.

A preferred reaction is the copolymerisation of 1-N acryloyl-1,2,3-benzotriazole with paraohloromethylstyrene as disclosed in the following example.

### EXAMPLE 3

Synthesis of the polymer by free radical polymerisation of N′-acryloyl-1,2,3-benzotriazole and p-chloromethylstyrene.

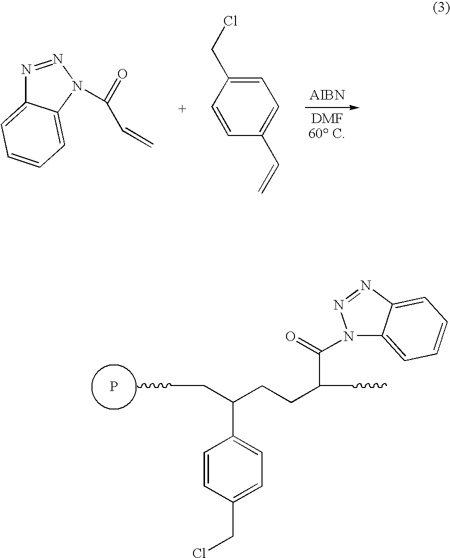

Into a carefully flamed Schlenk vial under nitrogen are added 1.156 mmol (200 mg) of N-acryl-1,2,3-benzotriazole and Li 156 mmol (176 mg) of p-chloromethylstyrene dissolved in 3 ml of anhydrous DMF and 4 mg of AIBN. The solution is subsequently transferred, by means of a needle under nitrogen pressure, into a round-bottomed polymerisation flask equipped With a magnetic stirring bar. The reaction is carried out at a temperature of 60° C. with constant stirring. After 20 hours, the polymer obtained, dissolved again in DMF, is precipitated in ethyl ether and filtered.

More generally, the polymer formation reaction may involve monomers bearing the various above mentioned leaving groups and occur under different solvent and temperature conditions.

As mentioned above, the polymers according to the invention are capable of reacting with the keratin amine groups at room temperature and under mild conditions, as shown in the following general reaction scheme for anchoring the polymer to hair, in relation to the embodiment where nucleophilic substitution is performed:

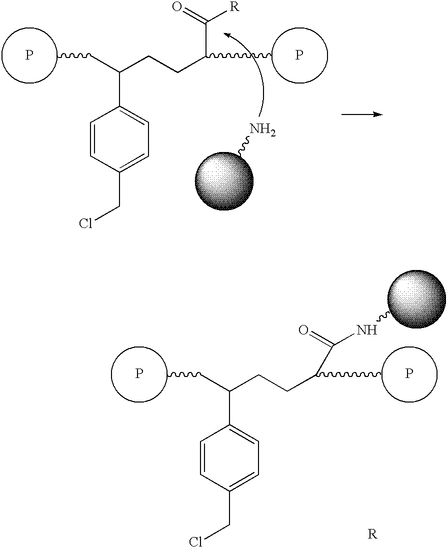

In the following in vitro test (example 4B), the above indicated reactions have been carried out using the polymer of formula 3 synthesised according to the details reported above.

### Example 4A

Application (in vitro) Tests—Polymer-Keratin Thiol Group Reaction

A solution of the polymer of formula 2 is prepared by dissolving 225 mg in 50 ml of DMF.

The formation of reversible polymer-keratin covalent bonds then proceeds as follows.

100 mg of hair is dispersed in 25 ml of DMF. 5 g of thioglycolic acid have been added, and the suspension left stirring for 20′ at room temperature in order to open the —S—S— bonds. The hair is filtered, and the filtered hair dispersed in the previously prepared polymer solution in DMF. 10 ml of hydrogen peroxide dispersion at 20 vol. have been added to the solution, leaving it to react for 15′ at room temperature.

The hair has subsequently been filtered and rinsed with DMF, and then water, and then dried.

### Example 4B

Application tests—Polymer-keratin amine group reaction. A solution of the polymer of formula 3 is prepared by dissolving 225 mg in 50 ml of DMF.

Subsequently, 25 ml of this solution are poured into a 100 ml round-bottomed flask containing 100 mg of hair. The desired reaction is that between the free amine groups on the keratin with the carbonyl groups present in the polymer. This reaction leads to the formation of new amide bonds with the liberation of 1,2,3-benzotriazole, according to the following reaction scheme.

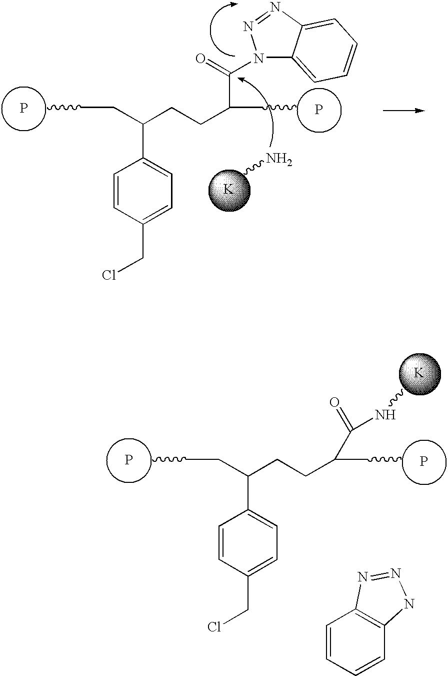

wherein K indicates the keratin structure and P indicates the polymer structure.

The reaction has been carried out for 5 hours at room temperature. Upon completion of the reaction, the hair thus treated has been filtered and washed with 50 ml of DMF to eliminate any residual polymer and the benzotriazole formed from the reaction, and well dried. Evidence that the reaction has occurred is provided by analysis of the UV spectrum following separation of the DMF filtrate by HPLC, since the benzotriazole i.e. the leaving, group liberated from the reaction, is contained in it.

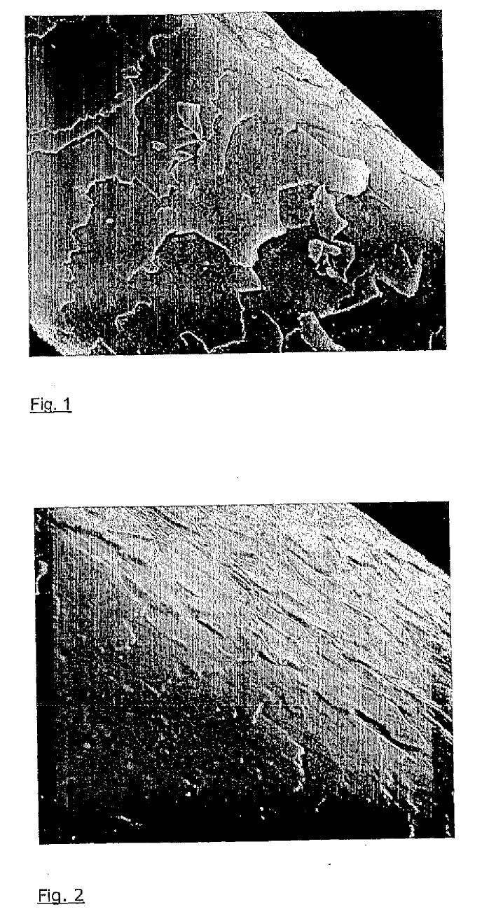

Scanning electron microscope analysis provides important data: it is clearly observed that the hair keratin structure has been coated with the polymer molecule. Scanning electron microscope analysis certainly gives important data: from FIGS. 1 and 2 it is clearly observed that the hair keratin structure has been coated with the polymer molecule. The same analysis has been performed on hair treated with DMF alone, in order to ascertain that this solvent does not interfere with the hair structure.

As further evidence of the reaction between the keratin amino groups and the polymer structure leaving groups, the determination of the free amino groups on the keratin has been performed following reaction with the polymers of the invention, according to the Guar and Gupta method. The Gaur and Gupta method for the determination of terminal amino groups envisages two stages, i.e. acylation of the amino groups using SDTB (N-succinimmidyl-4-O-(4,4′-dimethoxytriphenylmethyl)butyrate and the subsequent detritylation with acids.

### Acylation.

In a 10 ml round-bottomed flask fitted with a magnetic stirrer, a few mg of hair are made to react with 250 μl of reagent A, consisting of a solution of 0.5 mmol of SDTB in 5 ml of DMF. To the solution are added 5 ml of anhydrous triethylamine, as solvent, and a few crystals of dimethylaminopyridine (DMAP) to act as a catalyst for the reaction. Stirring is initiated and the mixture left to react for two hours.

The free amino groups on the keratin bind to the acylating agent according to the following reaction scheme:

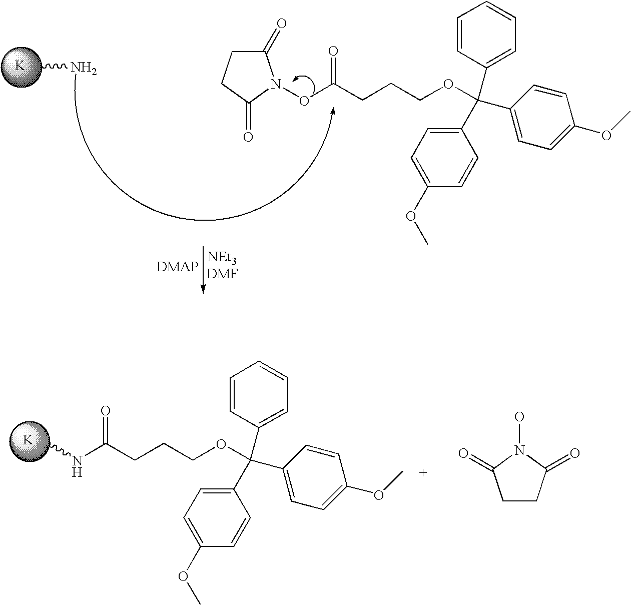

Upon completion of the reaction, the hair is washed with DMF, methanol, THF and diethyl ether in sequence, and then dried.

### Detritylation

Into a 10 ml flask are placed 21.71 mg of SDTB acylated hair, precisely weighed. Into said flask is added, up to a final volume of 10 ml, a detritylation solution consisting of HClO₄ and MeOH for the removal of the 4,4′-dimethoxytrityl (DMT) cationic group according to the reaction scheme:

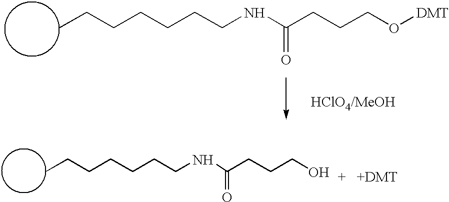

The dimethoxytrityl cations make the solution take on an orange colouration, and so the concentration of said ions may be calculated by UV spectrophotometry analysis. Said cations absorb at a wavelength of 498 nm. Once the absorbance of the trityl cations in the analysed sample has been analysed, it is then possible to calculate the moles of amino groups per gram of support, using the following formula:

$$\frac{moles \cdot of \cdot NH 2 groups}{grams \cdot of \cdot support} = \frac{A \cdot V}{P \cdot 70000}$$

where P is the weight in mc of the support subjected to detritylation, V is the volume in ml of the detritylation mixture where the polymer is introduced,

the hair treated with the polymer, as in example 4, is introduced

A is the absorbance measured and 7000 is the extinction coefficient of the trityl cation at 498 nm.

From the UV spectrophotometry analysis, the absorbance of the sample, thus prepared, is 0.058. The number of moles of amine groups per gram of support are thus calculated to be equal to 3.816·10⁻⁷, significantly different from the value measured for untreated hair. The same test performed on untreated hair gave a value of 8.2 10⁻⁶.

The process and the polymers according to the invention are hence capable of providing an exceptional method of treating hair, and keratin structures in general, allowing the binding of the polymer to the keratin by means of an actual bond, so as to obtain greater lasting duration over time, and so as to be able to use the hair-bound polymer as a receiving system for any additional compounds such as dyes, volumisers and other functional compounds, or protective agents, such as for example, antioxidants and antibacterial agents.

1. Polymers and copolymers having a functionalised polymeric structure in accordance with a general formula of 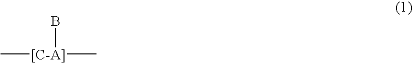
wherein [C-A] is a (co)polymer chain or a polymer chain selected from the group consisting of silicone, polyglycol, polysaccharide polymer structure or a natural polymer,
and B is a functional group selected from the group consisting of thiol groups or leaving groups within a side chain which, at room temperature and under reaction conditions compatible with use on humans, may form covalent bonds with keratin.

2. The polymers and copolymers according to claim 1,
wherein A is an acrylic, methacrylic, itaconic, sarcosinic or silicone residue; B is —SH or a leaving group selected from aromatic amines, 4-aminobiphenyl, benzidine, 4-chloro-o-toluidine, 2-naphthylamine, o-amino-azotoluol, 2-amino-4-nitrotoluol, p-chloroaniline, 2,4-diaminoanisole, 4,4′-diaminodiphenylmethane, 3,3′-dichlorobenzidine and 1,2,3 benzotriazole; and
C is a monomer selected from acrylic and methacrylic acid, itaconic acid, and the methyl or ethyl esters thereof with a C₃-C₃₀ alkyl group, styrene, styrene with halogen groups in the o,m,p positions, vinyl chloride, vinyl acetate, acrylamide, N,N dimethylacrylamide, N,N-diethylacrylamide, acrylamide substituted with C₃-C₃₀ alkyl groups, vinyl pyrrolidone, vinylpyrrolidinone, butadiene, cyanoacrylates and cyanomethacrylates substituted with C₃-C₃₀ alkyl groups, furoic acid, vinyl carbazole, vilidene chloride, vinyl alcohol, chloroprene, cyclopentadienes, terpenes and modified terpenes, or a linear or cyclic silicone structure.

3. The polymers and copolymers according to claim 2, wherein A is an acrylic or methacrylic residue, B is 1,2,3-benzotriazole and C is a monomer selected from the group consisting of acrylic and methacrylic or itaconic acid, and the methyl or ethyl esters thereof with a C₃-C₃₀ alkyl group, styrene, styrene with halogen groups on the o,m,p positions, vinyl chloride, vinyl acetate, acrylamide, N,N dimethylacrylamide, N,N-diethylacrylamide, acrylamide substituted C₃-C₃₀ alkyl groups, vinylpyrrolidone, vinylpyrrolidinone, butadiene, cyanoacrylates and cyanomethacrylates substituted with C₃ to C₃₀ alkyl groups, vinyl carbazole, vilidene chloride, vinyl alcohol, chloroprenecyclopentadienes, terpenes and modified terpenes.

4. The polymers and copolymers according to any one of the claims 1 to 3, characterised in that A-B has a general formula of 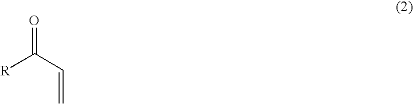
wherein R is selected from the group consisting of: chloride, bromide, iodide, tosylates (and the family thereof), carbonylimidazole derivatives, dicyclohexylurea derivatives (and the family thereof), 2-chloropyridinium ion derivatives, 3-chloroisoxatolidinium ion derivatives, 2,2′-dipyridyldisulphide derivatives, disulphurodimidazole, hydroxysuccinimide, aromatic amines, 1,2,3-benzotriazole;
and that C has a reactive double bond for free radical polymerisation.

5. The polymers and copolymers according to claim 4, wherein A-B is N′-acryloyl-1,2,3-benzotriazole and C is p-chloromethylstyrene.

6. A monomer for the synthesis of the polymers and copolymers according to claims 1-3 having a general formula of 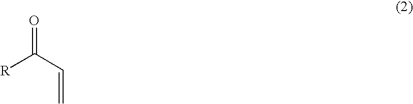
wherein R is selected from: chloride, bromide, iodide, tosylates (and the family thereof), carbonylimidazole derivatives, dicyclohexylurea derivatives (and the family thereof), 2-chloropyridinium ion derivatives, 3-chloroisoxatolidinium ion derivatives, 2,2′-dipyridyldisulphide derivatives, disulphurodimidazole, hydroxysuccinimide, aromatic amines, 1,2,3-benzotriazole.

7. A process for the synthesis of the polymers and copolymers according to claim 3, characterised in performing the free radical polymerisation of the monomer of general formula 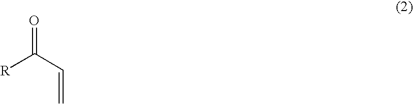
according to claim 3 with a monomer C having a reactive double bond for free radical polymerisation.

8. The process according to claim 7, wherein said monomer A-B is N′-acryloyl-1,2,3-benzotriazole and said monomer C is p-chloromethylstyrene.

9. The polymers and copolymers according to one of the claims 1 to 3, characterised in being functionalizable or functionalized with dyeing agents, volumising agents, antioxidants, lubricants, film forming agents, antibacterial agents.

10. A method of use of the polymers and copolymers according to claim 1, comprising the step of treating a keratin structure, the keratin structure being selected from the group consisting of hair, body hair, skin, nails, mucosa, lips and external genital organs.

11. A cosmetic formulation comprising one or more of the polymers and copolymers according to claim 1 in combination with cosmetically acceptable excipients, solvents and carriers.

12. The cosmetic formulation according to claim 11, wherein said solvents are polar solvents selected from: water, DMF, dioxan, dimethylacetamide, water in various concentrations of polyethyleneglycol (PEG) and mixtures thereof.

13. A dermatological formulation comprising one or more of the polymers and copolymers according to claim 1 in combination with pharmaceutically acceptable excipients, solvents and carriers.

14. A method for treatment with a cosmetic formulation according to one of claims 11 or 12 of keratin structures, the keratin structures being selected from the group consisting of hair, body hair, skin and nails, characterised in applying to them the cosmetic formulation to covalently bind the (co)polymer(s) contained in said formulation to the —SH or —NH₂ groups of said keratin structures.

15. The method according to claim 14, comprising subsequently applying dyeing agents, volumising agents, antioxidants, lubricants of film forming agents capable of reacting with functional groups present on said (co)polymer.

16. The method according to claim 14, comprising applying reducing agents, in order to open the disulphide bridges of the keratin structures, prior to application of the polymeric formulation.

17. The method according to claim 16, comprising applying oxidizing agents following said application of the polymeric formulation in order to create disulphide bridges between said polymer and said thiol groups.

18. (canceled)

19. (canceled)
